(19) United States
(12) Patent Application Publication
(10) Pub. No.: US 2017/0143254 A1
(43) Pub. Date: May 25, 2017

(54) ADJUSTABLE SMART PILLOW

(71) Applicant: JAB Distributors, LLC, Wheeling, IL (US)

(72) Inventors: Jared Bell, Northbrook, IL (US); Jose Miguel Marrero, Niles, IL (US); Warrick Bell, Northbrook, IL (US)

(21) Appl. No.: 14/948,660

(22) Filed: Nov. 23, 2015

(51) Int. Cl. A61B 5/00; A61B 5/11; A47G 9/10

(52) U.S. Cl. CPC A61B 5/4815; A47G 9/10; A47G 9/1036; A61B 5/11; A61B 5/6892; A47G 2009/1018

(57) ABSTRACT

An adjustable pillow including an outer shell, an inner top layer and an inner lower layer, a flexible bladder located between the inner top and lower layers, a closure mechanism located in the pillow that can be used to open and close an opening in the pillow, and a plurality of soft support members that can be inserted into and removed from the bladder. Each member includes a round core and a plurality of projections extending from the core. The pillow may also include a controller that communicates with a mobile device in order to monitor a user's sleep and provide sounds and movements that help the user fall asleep.

### BACKGROUND

Embodiments of the present invention generally relate to an adjustable pillow that can interact with the user to help the user fall asleep and track the user's sleep habits.

Conventional pillows come in different sizes, shapes, thicknesses, and levels of support. A single person may need several different pillows for different or changing sleeping conditions. For example, a person's sleep positions may change over time, e.g., a person may change from being a back sleeper to a side sleeper or stomach sleeper. Such changes in sleep position can be due to changes in the user's body shape and size due to weight fluctuations or pregnancy. A change in sleep position may lead to a change in the user's pillow preferences. For example, a side sleeper that changes to a stomach sleeper may go from preferring a thicker, fluffier pillow to a thinner, firmer pillow.

An adjustable pillow allows a user to adjust the thickness of the pillow to accommodate changes in pillow preferences. Existing adjustable pillows include a hole in the side of the pillow through which a user can remove or insert handfuls of fiber clumps or layers of foam. The process of removing fiber can be messy and unsightly. Moreover, the removed fiber clumps can easily come apart and portions of fiber can stick to other fabrics like clothes and carpet. In addition, it can be difficult to determine how much fiber to pull out or insert into the pillow in order to adjust the thickness of the pillow.

In addition, many people have trouble sleeping. Conventional pillows, however, do not typically provide users with much assistance in sleeping besides providing a soft surface upon which a user can place his or her head.

### SUMMARY

Certain embodiments of the present invention provide an adjustable pillow including an outer shell, an inner top layer and an inner lower layer, a flexible bladder located between the inner top layer and inner lower layer, a closure mechanism located in an opening in the pillow that can be used to open and close the opening, and a plurality of soft support members. Each member includes a round core and a plurality of projections extending from the core. The members are configured to be inserted into and removed from the bladder through the opening in the pillow to increase and decrease, respectively, the size of the pillow.

Certain embodiments of the present invention provide an interactive pillow including a pillow having an interior region and a controller in the interior region. The controller includes a processor, a speaker, a microphone, a motor, and a transceiver. The processor receives instructions via the transceiver to cause the speaker to generate sounds, cause the microphone to capture sounds, cause the motor to vibrate, and cause the sensor to detect movement.

Certain embodiments of the present invention provide a system for monitoring a user's sleep habits and activity habits. The system includes a web-enabled mobile device and a wearable activity tracker that includes a first transceiver and a first sensor. The first sensor detects a user's movement and the first transceiver communicates with the mobile device. The system includes a pillow having a controller with a second transceiver and a second sensor. The second transceiver communicates with the mobile device and the second sensor detects a user's movements. The tracker detects the user's movements over time while the user is awake and sends information related to the user's awake movements to the mobile device, and the mobile device provides the user with an activity report based on the awake movement information. The tracker and the pillow controller detect the user's movements over time while the user is asleep and send information related to the user's sleep movements to the mobile device, and the mobile device provides the user with a sleep report based on the sleep movement information.

Certain embodiments of the present invention provide a method for scoring a person's activity and sleep habits. The method includes providing a web-enabled mobile device, providing a wearable activity tracker that includes a first transceiver and a first sensor. The first sensor detects a user's movement and the first transceiver communicates with the mobile device. The method further includes providing a pillow including a controller having a second transceiver and a second sensor. The second transceiver communicates with the mobile device and the second sensor detects a user's movements. The method further includes downloading an application from a website with the mobile device in order to facilitate communication between the mobile device and the tracker and the mobile device and the pillow controller, detecting a user's movements over time while the user is awake with the first sensor of the tracker, sending the information related to the user's awake movements from the tracker to the mobile device via the first transceiver, and using the application on the mobile device to calculate an activity score for the user based on the awake movement information. The method further includes detecting a user's movements over time while the user is asleep with the first sensor of the tracker, sending the information related to the user's sleep movements from the tracker to the mobile device via the tracker first transceiver, detecting a user's movements over time while the user is asleep with the second sensor in the pillow controller, sending the information related to the user's sleep movements from the pillow controller to the mobile device via the second transceiver, and using the application on the mobile device to calculate a sleep score for the user based on the sleep movement information sent by the tracker and by the pillow controller. The method further includes using the application on the mobile device to calculate a total life score based on the awake movement information sent by the tracker and on the sleep movement information sent by both the tracker and the pillow controller, and displaying the activity score, the sleep score, and the total life score on a screen of the mobile device.

### BRIEF DESCRIPTION OF SEVERAL VIEWS OF THE DRAWINGS

FIG. 1 illustrates an isometric view of a pillow according to an embodiment of the present invention.

FIG. 2 illustrates a side view of the pillow of FIG. 1.

FIG. 3 illustrates a cutaway side view of the pillow of FIG. 1 taken along lines 3-3.

FIG. 4 illustrates a cutaway top view of the pillow of FIG. 2 taken along lines 4-4.

FIG. 5 illustrates a cutaway side view of a pillow that includes a controller according to an embodiment of the present invention.

FIG. 6 illustrates a block diagram of the components of the controller of FIG. 5.

FIG. 7 illustrates an isometric view of an activity tracker according to an embodiment of the present invention.

Before the embodiments of the invention are explained in detail, it is to be understood that the invention is not limited in its application to the details of construction and the arrangement of the components set forth in the following description or illustrated in the drawings. The invention is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION

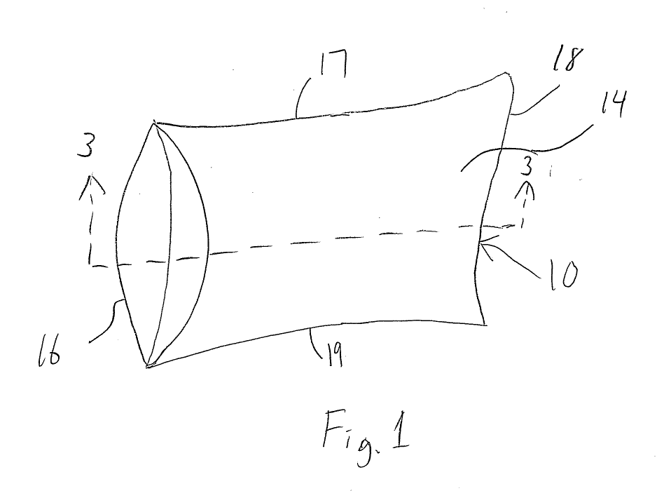

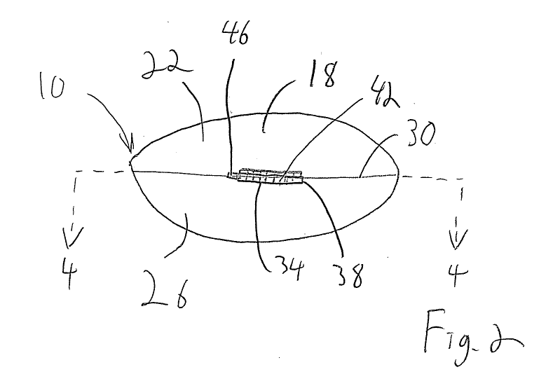

FIG. 1 illustrates an isometric view of a pillow 10 according to an embodiment of the present invention. The exterior of the pillow 10 is fabric. The pillow 10 includes first and second side ends 16 and 18 and first and second long ends 17 and 19. FIG. 2 illustrates a side view of the second side end 18 of the pillow 10. The pillow 10 has a top portion 22 and a bottom portion 26 separated by a seam 30. A closure system 34 is located along a hole 38 approximately in the middle of the seam 30. The closure system 34 includes zipper tracks 42 and a zipper head 46 that can be used to separate and join the zipper tracks 42 in order to open and close, respectively, the hole 38 in the seam 30. When the hole 38 is open, a user can insert his or her hand inside a cavity in the pillow 10. Alternatively, other closure mechanisms can be used besides a zipper system to open and close the hole. It will be appreciated that the closure system 34 can be located on either the first side end 16 or the second side end 18 of the pillow 10 or on both the first side end 16 and the second side end 18 of the pillow 10 or at other locations on the pillow 10.

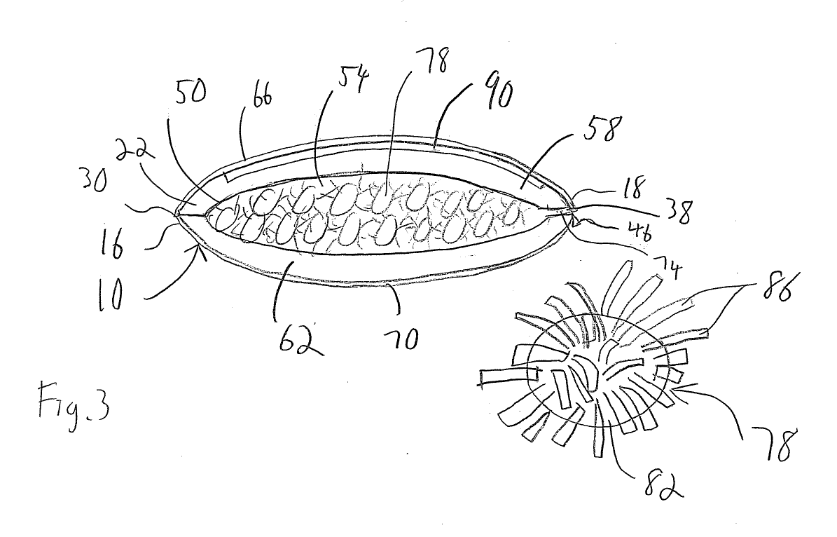

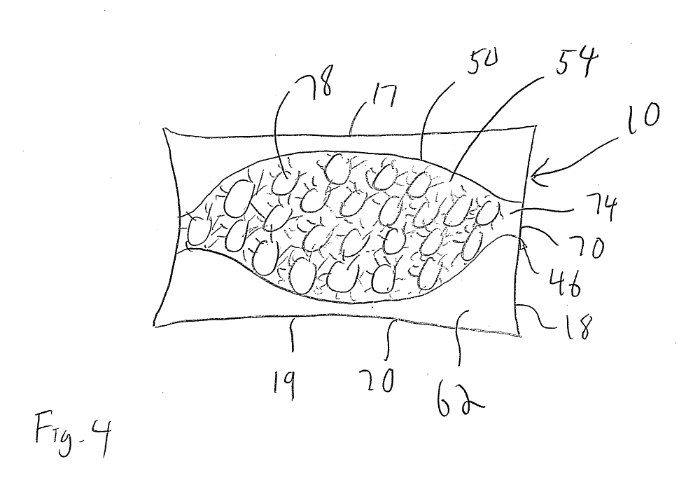

FIG. 3 illustrates a cutaway side view of the pillow 10 of FIG. 1 taken along lines 3-3, and FIG. 4 illustrates a cutaway top view of the pillow 10 of FIG. 2 taken along lines 4-4. The pillow 10 includes a flexible bladder 50 that defines a cavity 54. The bladder 50 can be made of fabric such as fiber knit polyester or any number of other flexible and/or soft materials. The bladder 50 is located between a top barrier or layer 58 and a bottom barrier or layer 62. The top layer 58 and bottom layer 62 are soft and can be made of memory foam. Alternatively, the top and bottom layers 58 and 62 can be made of any number of other soft materials such as a polyester filling or other types of fabric or micro fiber. A cooling gel top 90 is located on top of the top layer 58. The gel top 90 and the top layer 58 are surrounded by a top fabric shell 66, and the bottom barrier 62 is surrounded by a bottom fabric shell 70. The shells 66 and 70 are connected at seams 30 along the first and second side ends 16 and 18 and first and second long ends 17 and 19 of the pillow 10. The shells 66 and 70 can be made out of any number of different fabrics or textiles. The cavity 54 of the bladder 50 is connected to the hole 38 at the second side end 18 of the pillow 10 by a channel 74.

A number of support clusters or members 78 are located in the cavity 54 in the bladder 50. The support clusters 78 are soft and compressible. The size of the support clusters 78 can vary. Each cluster 78 includes a ball-shaped core 82 from which projections 86 extend. The projections 86 are soft and have a generally cylindrical shape but can have other shapes. The projections 86 on the same core 82 may have different lengths. The core 82 and projections 86 may be made of polyester or any number of other soft, flexible, and or compressible materials such as fabric, fiber, or foam.

In operation, a user can pull the zipper head 46 to open the hole 38 in the second side end 18 of the pillow 10. The user can stick his or her hand through the hole 38 and channel 74 and into the cavity 54 of the bladder 50 to insert or remove support clusters 78 from the bladder 50 in order to adjust the thickness or height of the pillow 10. By inserting clusters 78 into the bladder 50, the user can increase the thickness and/or firmness of the pillow 10, and by removing clusters 78 from the bladder 50, the user can reduce the thickness and/or firmness of the pillow 10. In this way, the user can adjust the thickness and firmness of the pillow 10 as desired to customize pillow support and accommodate changes in the user's sleep patterns or the user's body shape and size. Because the clusters 78 are generally the same size and shape, the user can measure how many clusters 78 he or she needs to add to or remove from a pillow 10 in order to adjust the pillow 10 to a desired thickness and support level. In this way, the user knows how many clusters 78 need to be in the bladder 50 for the pillow 10 to have the thickness and support qualities the user desires. Therefore, if the user has to change the number of clusters 78 in the pillow 10, such as to accommodate the support preferences of a guest who borrows the pillow 10, the user can easily adjust the pillow 10 back to the user's preferred thickness by inserting or removing clusters 78 until the appropriate number of clusters 78 for that thickness are in the bladder 50.

Moreover, because the pillow 10 is adjustable, the user only needs one pillow and does not need to buy or store numerous different kinds of pillows having different thicknesses and support levels should the user's support preferences change or should the user have guests who have pillow preferences that differ from the user's preferences. As such, the adjustable pillow 10 reduces the number of different kinds of pillows a person needs to have and the number of different kinds of pillows a retailer needs to make, carry, and store.

In addition, the cooling gel top 90 provides a cooling sensation to the user when the user places his or her head on the top portion 22 of the pillow 10. This cooling sensation helps users who are “hot” sleepers. Alternatively, for users who are not “hot” sleepers, the pillow 10 may not include the cooling gel top 90 at all. Alternatively, the pillow 10 may include multiple cooling gel portions located at different parts of the pillow 10, such as the top and bottom portions 22 and 26 of the pillow 10.

The pillow 10 of the various embodiments may be made of hypo allergenic and/or anti-bacterial materials.

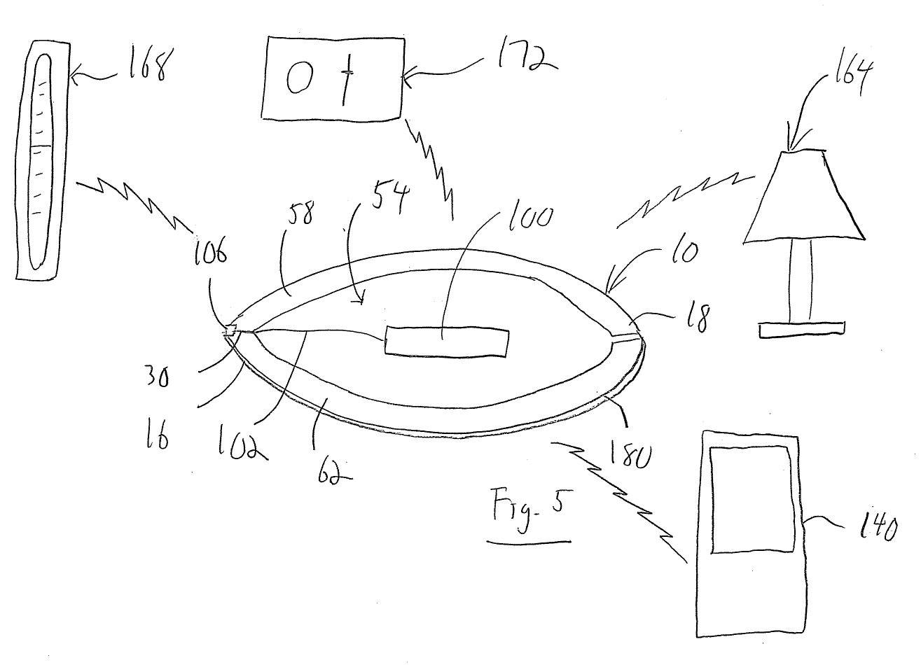

FIG. 5 illustrates a cutaway side view of an adjustable pillow 10 that includes a controller 100 located in the cavity 54 of the pillow 10. The controller 100 is configured to be fixed in place in the cavity 54 between the top and bottom barriers 58 and 62. Alternatively, the controller 100 can be removable from the cavity 54 by a closable opening in the exterior of the pillow 10 that is connected to the cavity 54. The pillow 10 can include a bladder and clusters like the pillow 10 shown in FIGS. 1-4. Alternatively, the controller 100 can be used in a conventional pillow.

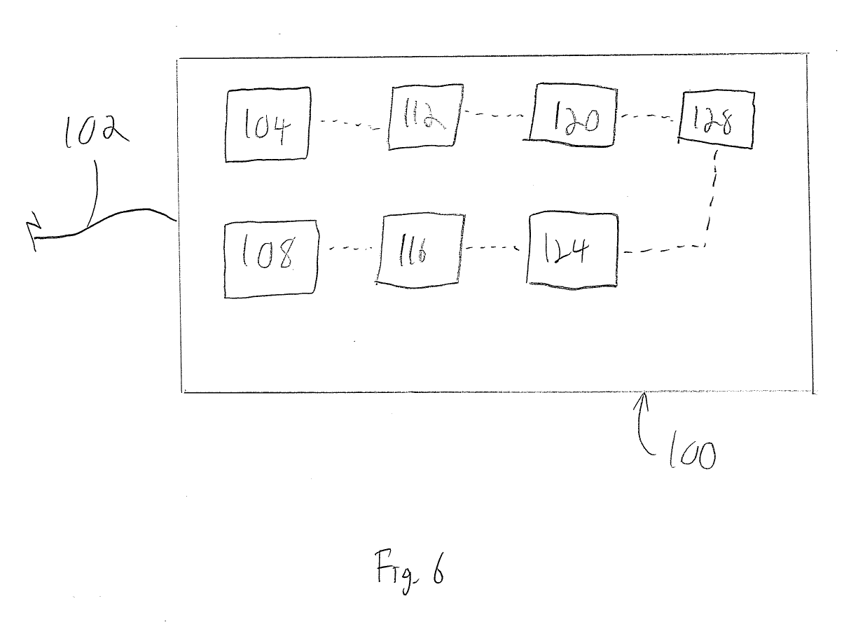

FIG. 6 illustrates a block diagram of the components of the controller 100. The controller 100 includes a microphone 104, a sensor or accelerometer 108, a speaker 112, a vibrating motor 116, a wireless transceiver 120, a power source such as a battery 124, and a processor/memory unit 128. By way of example, the transceiver 120 may be a Bluetooth™ device. In particular, the transceiver 120 may be a Bluetooth™ Audio and/or Bluetooth™ device. The microphone 104, accelerometer 108, speaker 112, vibrator 116, transceiver 120, and power source 124 are all connected to and in communication with the processor unit 128.

Returning to FIG. 5, the controller 100 is connected to a connector or cord 102 that extends to a charging and/or data port 106 located outside of the pillow 10. The connector 102 can include a power bus and data bus. The port 106 may be located along the seam 30 at one of the first and second side ends 16 and 18 of the pillow 10. The port 106 may include a USB port. The connector 102 delivers power and data to the controller 100. A computerized device 140, such as a smart phone, personal digital assistant device, smart device or tablet, or other kind of web-enabled mobile device, can be connected to the port 106 via a connector to communicate with the controller 100 and, more specifically, the processor 128 in the controller 100. In addition, a charging cord (not shown) connected to a conventional wall outlet or other power source can be connected to the port 106 to provide power to the controller 100 and charge the battery 124. A user can download software or a program, also known as an “application” or “app,” for the controller 100 in the pillow 10 from a website to the phone 140. The application allows the user to communicate with and control the pillow controller 100 through the phone 140 and allows the phone 140 to communicate with the website. The phone 140 is configured to communicate with the processor 128 of the controller 100 wirelessly via the transceiver 120. The phone 140 can also communicate with the processor 128 of the controller 110 via a direct connection between the phone 140 and the port 106 on the pillow 10.

The user can use the phone 140, through the application, to cause the controller 100 to perform a number of functions to help the user sleep, monitor the user's sleep, and provide feedback to the user about his or her sleep patterns. The pillow 10 can include piping 180 on the exterior thereof. The piping 180 can include a light source that causes the piping 180 to light up in order to signal to the user that the controller 100 is activated or set up to perform various selected functions.

In particular, a user can use the application on the phone 140 to instruct the processor 128 in the controller 100 to play a particular type of music through the speakers 112 for a particular amount of time. For example, if the user goes to bed at 10:00 p.m. and likes to listen to classical music to facilitate sleep, the user can use the phone 140 to program the controller 100 to start playing classical music on the speakers 112 at 10:00 p.m. and automatically stop playing the music at 10:30 p.m. The speakers 112 can be configured to provide sound in such a way that the user can only hear the sound when the user's head is on the pillow 10. In that regard, the speakers 112 function like bone conduction headsets that decode sound waves and convert them into vibrations and then transmit the vibrations against the side of head. The vibrations travel from the side of the head to the inner ear of the user via bone conduction. For example, if the user sleeps with the left side of his or her head on the pillow 10, and the speaker 112 is positioned proximate the user's head or jaw, the user will hear the music in his or her left inner ear. In this way, the music from the speakers 112 can only be heard by the user and does not disturb the user's partner who may be lying next to the user in bed. Alternatively, the speaker 112 can be a conventional audio speaker that sends sound waves directly to the ear drum. By incorporating the speakers 112 into the controller 100 in the pillow 10, a user does not have to wear headphones or ear buds in order to listen to music while falling asleep.

The user can also instruct the speaker 112 to play other audio content such as books on tape, podcasts, or other media. The audio content can be downloaded to the phone 140 from the Internet and then sent from the phone 140 to the controller 100. In addition, the speaker 112 can be instructed to play voice mails or read emails or text messages from the user's phone to the user while the user has his or her head on the pillow 10. For example, the speaker 112 can read a text message or email to the user either at the time the phone 140 receives the text message or email or at a time at which the user wishes to have all, or specific, texts or emails read to him or her.

The user can also use the phone 140 to program the controller 100 to cause the vibrating motor 116 to create a gentle vibration in the pillow 10 to help the user fall asleep. For example, the user can program the motor 116 to vibrate for ten minutes and then automatically shut off. Alternatively, the user can program the motor 116 to vibrate and the speaker 112 to play music at the same time for a certain period of time to help the user fall asleep.

In addition, the user can use the phone 140 to program the motor 116 to start vibrating at a specific time to wake the user up, or program the speaker 112 to play music at a specific time to wake the user up, or program both the speaker 112 and the motor 116 to operate at a specific time to wake the user up with music and vibration.

The controller 100 in the pillow 10 can also be configured to connect with other devices in a bedroom through the phone 140 and/or the wireless transceiver 120. For example, the controller 100 can be configured to communicate with a lamp or light 164 in the room that also includes a transceiver. The user can use the phone 140 to instruct the controller 100 to turn the light 164 on, turn it off, or adjust the brightness of the light 164 at specific times. For example, the user can program the controller 100 to cause the light 164 to turn off at a specific time to let the user know he or she should stop reading or watching television and go to sleep. Alternatively, the controller 100 can cause the light 164 to start dimming at a specific time and continue dimming over time until the light 164 is completely off at a particular time. The controller 100 can also cause the light 164 to turn on at a specific time in the morning to wake up the user. Alternatively, the controller 100 can cause the light 164 to begin to turn on at a very low brightness level at a specific time and gradually get brighter over a specific period of time in order to wake the user up. In this way, the light 164 can be controlled to simulate light entering a room during the rising of the sun in the morning.

The controller 100 can also be programmed, via the phone 140, to coordinate the activation of one of the light 164, speaker 112, and motor 116 with the activation of any or both of the others. By way of example only, the controller 100 can cause the light 164, speaker 112, and motor 116 to all activate at the same time in the morning such that the user is woken up by a combination of light from the light 164, sound from the speaker 112, and vibration from the motor 116.

The controller 100 in the pillow 10 can also be configured to wireles sly communicate, through the phone 140 and/or through the transceiver 120, with a thermometer 168 and a temperature changing device 172, such as a thermostat, fan, and/or heater. The user can use the phone 140 to instruct the controller 100 to periodically communicate with the thermometer 168 to monitor the temperature of the room in which the user is sleeping. If the room temperature is not at a desired temperature at a specific time, the controller 100 can activate the thermostat, fan, or heater 172 to adjust the temperature in the room to the desired temperature. For example, a user that sleeps “hot” may find that he or she is comfortable at a particular temperature when getting into bed at 10:00 p.m. but wakes up at 1:00 a.m. feeling hot at that same temperature. Accordingly, the user can use the phone 140 to program the controller 100 to communicate with the thermometer 168 and thermostat 172 in order to get the room to a desired temperature at the time the user goes to bed, e.g., 10:00 p.m. The controller 100 can then communicate with the thermometer 168 and thermostat 172 to lower the temperature of the room by a degree by 1:00 a.m. so that the user does not wake up “hot” at 1:00 a.m.

The user can also use the phone 140 to instruct the controller 100 to monitor aspects of the user's sleep patterns. The controller 100 can be instructed to begin monitoring sleep patterns at specific times, such as, by way of example only, between 10:00 p.m. and 6:00 a.m. Alternatively, the controller 100 can time the monitoring of sleep habits with respect to other functions performed by the controller 100. For example, the controller 100 can begin monitoring the user's sleep patterns right after the speaker 112 stops playing music and/or the light 164 goes completely off.

One way the controller 100 can monitor a user's sleep patterns is to monitor the user's snoring habits. The controller 100 can activate the microphone 104 for pre-selected periods when the user is sleeping. The microphone 104 can detect the user snoring and send that information to the processor 128 and/or phone 140. The phone 140 or processor 128 can then activate the motor 116 in the pillow 10. The motor 116 gently vibrates just enough to cause the user to adjust the position of his or head without waking the user up. The change in head position can open up the user's breathing passages such that the user stops snoring. The processor 128 and phone 140 can also store information related to when and how long the user snores while the user is asleep.

A user can also use the phone 140 to instruct the controller 100 to monitor the user's movements while asleep. For example, the processor 128 can cause the sensor/accelerometer 108 to detect and record movement of the user's head and/or neck while the user sleeps. The accelerometer 108 can send that information related to movement (such as when and how much movement occurred) to the processor/memory 128, and the processor 128 can send the information via the transceiver 120 to the phone 140.

The phone 140 can use the application to analyze the information related to a user's snoring and movement and prepare a report for the user regarding the user's sleep patterns. For example, information related to movement over time and/or snoring can be used to determine when and for how long the user was in light sleep, deep sleep, REM sleep, or awake at certain times during the course of the night. Moreover, the phone 140 can communicate with the thermometer 168 to check the room temperature during the course of the night and evaluate whether changes in room temperature affect or correspond to changes in the user's sleep patterns.

Thus, the phone 140 can use the movement, snoring, and temperature data to evaluate a user's sleep. The phone 14 can prepare a sleep report based on the evaluation of those factors, and the user can view the report on the phone 140 when the user wakes up in the morning. The report can include a graph depicting sleep quality, and a breakdown showing how much time the user spent awake, in light sleep, in deep sleep, and in REM sleep. The report can also include a sleep score based on the type of sleep and duration of sleep the user had through the course of the night. Alternatively, the user can view the report on another computerized device to which the phone can send the report by email or text.

More particularly, as part of preparing the sleep report, the phone can use the application to assign points to the quality of a user's sleep through the night to arrive at a sleep score. There are a number of ways sleep points and a sleep score can be calculated. For example, the points and score could be based on a number of different factors such as the number of times the user was in deep sleep, in REM sleep, or in light sleep, the duration of the deep sleep, REM sleep, or light sleep periods, the amount of time the user was awake, etc. For example, deep sleep may be sleep with no movements over a particular time period, REM sleep may be sleep with only a few movements over that time period, and light sleep may be sleep with more than a few movements in that time period. An awake state may be defined as numerous movements or constant movement in the time period. A number of points may be assigned for each minute a user is in one of the deep sleep, light sleep, REM sleep, or awake states. The number of points assigned for each minute a user is in a particular sleep state may vary with the sleep state. For example, a minute of deep sleep may receive more points than a minute of REM sleep, which may receive more points than a minute of light sleep. Each minute of an awake state may receive no points.

Additionally, or alternatively, a user may be given points for sustained sleep in a particular state. For example, a user may receive a number of additional points for a particular number of minutes of sustained deep sleep, a number of additional points for a particular number of minutes of sustained REM sleep, and a number of additional points for a particular number of minutes of sustained light sleep. The points for the sustained period of deep sleep may be more than the points for the sustained period of REM sleep, which may be more than the points for the sustained period of light sleep. After the user's total sleep period is over, the phone 140 calculates a total sleep score by adding the number of points the user received during the course of the night for his or her deep sleep, light sleep, and REM sleep. By way of example, a total score over a certain threshold amount, e.g., 300 “sleep points,” means that the user got enough good sleep, and a score under 300 sleep points means the user did not get enough good sleep. If the user consistently has a score under 300 sleep points, the user can use that information to determine if he or she needs to adjust his or her sleep habits to get better sleep. The user can use the sleep scores to try to target new sleep goals or alter sleep patterns so that the user can get more and better sleep.

Besides a total sleep score, the phone 140 can also calculate and provide to the user information regarding when a user was in a particular state of sleep (e.g., deep sleep, REM sleep, or light sleep), the total amount of time the user was in a particular state of sleep, and the amount of points the user received for a particular state of sleep. The phone 140 can also provide the user with information regarding the total amount of time the user was asleep regardless of which sleep state the user was in.

In addition, the phone 140 can store the nightly sleep report information over a period of time. Alternatively, the phone 140 can send the sleep reports to the website from which the application was downloaded, and the proprietor of the website can create a record of a user's sleep reports. For example, using saved sleep reports, the phone 140 can present a summary of seven nights' worth of sleep reports every week to the user. The summaries can provide information in charts, scores, and graphs. The user can use the sleep reports and summaries to understand how well the user slept over time.

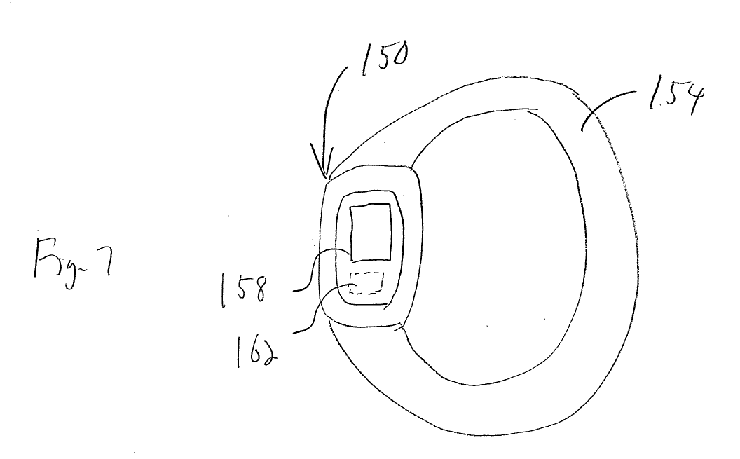

In addition, and with reference to FIG. 7, the pillow 10 can be used in conjunction with an activity tracker 150 to monitor a user's sleep movement and assign sleep points to a user for the previous night sleep. The tracker 150 includes a wrist band 154 so that the tracker 150 can be worn on the user's wrist as the user sleeps. The tracker 150 can also be configured to be worn on other parts of a user's body or on a user's belt or other article of clothing. The tracker 150 includes a screen 158 and sensors 162 that detect movement. For example, the tracker 150 can include a pedometer and/or an accelerometer. The tracker 150 is configured to communicate with the user's phone 14 via wireless communication such as Bluetooth™ technology. In that regard, the tracker 150 includes a wireless transmitter/receiver.

In operation, and with respect to FIGS. 5-7, a user wears the tracker 150 when the user goes to sleep on the pillow 10. While the user sleeps, the sensor 108 in the pillow 10 monitors the user's head movements, and the sensor 162 in the tracker 150 likewise tracks the user's wrist and/or arm movements. Both the pillow controller 100 and the tracker 150 send the movement information to the phone 140. The phone 140 can then use the movement information to prepare a sleep report and sleep score.

Alternatively, a user could wear more than one tracker 150 while sleeping. For example, the user can wear a tracker 150 on each arm and/or on each leg. Each of the multiple trackers 150 sends movement information for the body part to which the tracker 150 is attached to the phone 140 for incorporation into the sleep report. This results in a more detailed sleep report based on more movement information.

The controller 100 in the pillow 10 and the tracker 150 can also be used with the application on the phone 140 and the website to analyze both a user's sleep habits at night and a user's daytime activity habits when the user is awake. In that regard, the tracker 150 can have two different settings: a sleep setting and an activity setting. Before going to sleep the user can use the screen 158 to put the tracker 150 on the sleep setting, and when the user wakes up, the user can use the screen 158 to put the tracker 150 on the activity setting.

The sensor 162 of the tracker 150 can be programmed to track, over the course of a day, user activity information such as steps over time, the distance the user walks, the user's heart rate and/or pulse, and calories the user burns. Calculation of calories burned can be based on personal information (such as age, weight, and height) the user supplies to the tracker 150, phone 140, or website from which the application is downloaded. The tracker 150 can send the user's activity information to the phone 140 and, using the application, the phone 140 uses that activity information to create an activity report for the user for the day. In addition, the tracker 150 can provide an activity report or score based on the activity information it tracks and show that score to the user on the screen 158.

In one embodiment, the activity report can include activity points and an activity score. There are a number of ways sleep points and a sleep score can be calculated. The points and score can be based on a number of different factors such as number of steps, calories burned, heart rate, the duration and intensity of the user's movements, etc. For example, the phone 140 can assign points depending on how intense and sustained a user's particular activities are. The more sustained and intense the user's activities are, the higher the user's daily activity score is. At the end of the day, the phone 140 can add up or combine the points related to the user's activities over the course of the day and provide a final active score for the day. In addition, the tracker 150 can provide activity scores to the user during the course of the day for various activities the user engages in or show the user what his or her activity score for the day is at a particular point in time.

Activity points may be assigned and tallied a number of different ways. In one example, a user's activities may be broken down into five categories based on the number of steps the tracker 150 determines the user takes in a particular period of time. Those categories may include a resting state, a walking state, an active state, a very active state, and an extremely active state. Each state may be defined by a range of the number of steps a person takes in a particular time period. A user may receive a certain number of points for every minute the user is in a particular state. For example, the user may not receive any points for time the user is in the resting state or walking state but may receive a number of points for each minute the user is in the active state, the very active state, and the extremely active state. The user may receive more points for each minute the user is in the extremely active state than the user receives for each minute the user is in the very active state, and may receive more points for each minute the user is in the very active state than the user receives for each minute in the active state.

Additionally, or alternatively, the user may receive points for a sustained amount of time in a particular state. For example, the user may receive a certain number of points for every 15 minute period the user is in one of the active, very active, or extremely active states. The user may get more points for being in the extremely active state for the 15 minute period than for being in the very active state for the 15 minute period. Similarly, the user may get more points for being in the very active state for the 15 minute period than for being in the active state for the 15 minute period.

Thus, for example, User A may work in an office, and the tracker 150 may track User A taking 2,100 steps over the course of an 8 hour work day in the office. User A, however, may get only 5 activity points for that activity. On the other hand, User B may walk 2,000 steps over a 20 minute period to get to the bus to go to work in the morning. User B may get 100 active points for that activity. Though User B took fewer total steps than User A, User B received a higher activity score for the activity. This is because User A's activity was intermittent and spread out through the day while User B's activity was more sustained and intense. In that regard, User B took almost as many steps as User A but in a much shorter time period. As another example, the tracker 150 and phone 140 can be used to assign 100 activity points to a user for one hour of sustained walking, 45 minutes of sustained jogging, or 30 minutes of sustained running.

At the end of the day and after the user's active period is over, the phone 140 can communicate with the tracker 150 to calculate a total active score by adding the number of points the user received during the course of the day for his or her active states, very active states, and extremely active states. A total score over a certain threshold or goal amount, e.g., 300 “active points,” can mean that the user got enough activity, and a score under 300 active points can mean the user did not get enough activity for the day. The 300 point goal could correspond to, by way of example only, one hour of vigorous sustained walking. If the user consistently has a score under 300 active points, the user can use that information to make more of an effort to be active during the day in order to reach a score of 300 active points each day.

Besides a total activity score, the phone 140 can also provide an activity report to the user detailing when during the day the user was in a particular activity state (e.g., extremely active very active, or active), the total amount of time the user was in a particular activity state, and the amount of points the user received for a particular activity state for the day.

In addition, the user can track his or her activity points in real time over the course of the day to make sure he or she engages in enough activity to reach the goal by the end of the day. Thus, if toward the end of the day, a user can see from the tracker 150 or phone 140 that he or she needs 100 more activity points to reach the goal of 300 points, the user can go for a walk or run to get to the goal. In addition, the tracker 150 or phone 140 can also classify the kinds of activity the user engages in during the day in real time so the user knows how intense his or her activities are. For example, the phone 140 and/or tracker 150 can inform that the user that 30 minutes of running was “extremely active,” 45 minutes of light jogging was “very active,” and one hour of walking was “active.” In this way, the user can know how rigorous his or her activities are during the day.

The phone 140 can also be used to combine and analyze both a user's sleep score and activity score to create a daily life score for the user. In particular, the tracker 150 can provide information to the phone 140 to create an activity score for the day, the pillow 10 and the tracker 150 can provide information to the phone 140 to create a sleep score, and the phone 140 can evaluate and combine the sleep score and activity score to provide the user with a life score for the past day. Thus, the calculation of the daily life score is based on a combination of the user's active points from the day and the user's sleep points from the night before.

By way of example only, the life score can be calculated by weighting a user's active points score and sleep points score by multiplying each score by a particular variable and then adding the weighted scores together. In addition, or alternatively, before being added, each weighted active score and sleep score can be capped so as not exceed a certain number. Similarly, the life score can be capped so as not to exceed a certain number. A life score goal of a certain amount, e.g., 360 life points, can mean that the user had a combination of a proper amount of activity and sleep during the course of the day.

By further example, the life score may be calculated such that a user may not be able to obtain 360 life points if he or she has a very high activity score but a very low sleep score. Similarly, a user may not be able to obtain 360 life points even if he or she has a very high sleep score if his or her activity score is very low. In addition, a user may have to obtain a minimum number of activity points and sleep points to reach 360 life points. A user can check his or her life score every day to determine if he or she is meeting his or her goals for a proper balance between daily activity and nightly sleep. If the user does not meet his or her sleep score, activity score, or life score goals, the user can use this information to adjust activity and sleep habits in order to improve his or her scores.

The life score can be shown on the phone 140 but it also can be sent to other computerized devices or accessed from the application provider's website. The activity scores, sleep scores, and life scores can be plotted over time on a report or chart so that the user can see his or her scores over time. The information related to a user's scores can be stored in the cloud, on the phone 140, or on another computerized device.

While the activity score can be weighted to give more points to more intense and/or sustained activities over time, it will be understood that activity points can also be weighted in different ways to emphasize different aspects of a user's activities. Similarly, while the sleep score can be weighted to give more points to deep sleep and/or sustained deep sleep over time, it will be understood that sleep points can also be weighted in different ways to emphasize different aspects of a user's sleep patterns.

While various spatial and directional terms, such as top, bottom, lower, mid, lateral, horizontal, vertical, front and the like may used to describe embodiments of the present invention, it is understood that such terms are merely used with respect to the orientations shown in the drawings. The orientations may be inverted, rotated, or otherwise changed, such that an upper portion is a lower portion, and vice versa, horizontal becomes vertical, and the like.

Variations and modifications of the foregoing are within the scope of the present invention. It is understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

1. An adjustable pillow, comprising:
an outer shell;
an inner top layer and an inner lower layer;
a flexible bladder located between the inner top layer and inner lower layer;
a closure mechanism located in an opening in the pillow that can be used to open and close the opening;
a plurality of soft support members, wherein each member includes a round core and a plurality of projections extending from the core, the members being configured to be inserted into and removed from the bladder through the opening in the pillow to increase and decrease, respectively, the size of the pillow.

2. The adjustable pillow of claim 1, wherein the pillow includes a cooling gel portion along the inner top layer.

3. The adjustable pillow of claim 1, wherein the inner top layer and inner bottom layer are made of memory foam.

4. The adjustable pillow of claim 1, wherein the support member is made of fabric.

5. The adjustable pillow of claim 4, wherein each support member includes projections having different lengths.

6. The adjustable pillow of claim 1, wherein the closure mechanism comprises a zipper and zipper tracks.

7. The adjustable pillow of claim 1, further including a speaker.

8. The adjustable pillow of claim 7, wherein the speaker transmits sound via bone conduction.

9. The adjustable pillow of claim 1, further including a controller that includes a speaker, a transceiver, a processor, a motor, a sensor, and a microphone.

10. The adjustable pillow of claim 9, wherein the controller communicates with a web-enabled mobile device via the transceiver, and the mobile device can cause the speaker to plays sounds, the motor to vibrate, the sensor to detect movement, and the microphone to capture sounds.

11. An interactive pillow, comprising:
a pillow having an interior region;
a controller in the interior region, the controller comprising a processor, a speaker, a microphone, a motor, and a transceiver;
wherein the processor receives instructions via the transceiver to cause the speaker to generate sounds, cause the microphone to capture sounds, cause the motor to vibrate, and cause the sensor to detect movement.

12. The pillow of claim 11, wherein the controller receives instructions from a web-enabled mobile device via the transceiver.

13. The pillow of claim 11, further including a port connected to the controller by a connector, wherein the port can receive power and data signals.

14. The pillow of claim 11, further including piping that illuminates.

15. The pillow of claim 11, wherein the speaker transmits sound via bone conduction.

16. The pillow of claim 11, wherein the controller is configured to communicate with a light via the transceiver to activate or deactivate the light.

17. The pillow of claim 11, wherein the controller is configured to communicate with a thermometer via the transceiver to track changes in room temperature.

18. The pillow of claim 11, wherein the controller activates the microphone to detect snoring and, if snoring is detected, the controller activates the motor to vibrate.

19. The pillow of claim 11, wherein the controller causes the sensor to detect movement of a user's head and sends information related to the movement to a computerized device via the transceiver.

20. The pillow of claim 19, wherein the computerized device is a web-enabled mobile device and uses the information related to movement to create a sleep report for the user.

21. The pillow of claim 20, wherein the computerized device presents the sleep report as a score.

22. The pillow of claim 11, further including:
an inner top layer and an inner lower layer;
a flexible bladder located between the inner top layer and inner lower layer;
a closure mechanism located in the pillow that can be used to open and close an opening in the pillow;
a plurality of soft support members, wherein each support member includes a round core and a plurality of projections extending from the core, and the support members are configured to be inserted into and removed from the bladder through the opening in the pillow to increase and decrease, respectively, the size of the pillow.

23. A system for monitoring a user's sleep habits and activity habits, comprising:
a web-enabled mobile device;
a wearable activity tracker that includes a first transceiver and a first sensor, wherein the first sensor detects a user's movement and the first transceiver communicates with the mobile device;
a pillow including a controller having a second transceiver and a second sensor, wherein the second transceiver communicates with the mobile device and the second sensor detects a user's movements; and wherein:
the tracker detects the user's movements over time while the user is awake and sends information related to the user's awake movements to the mobile device, and the mobile device provides the user with an activity report based on the awake movement information; and
the tracker and the pillow controller detect the user's movements over time while the user is asleep and sends information related to the user's sleep movements to the mobile device, and the mobile device provides the user with a sleep report based on the sleep movement information.

24. The system of claim 23, wherein the activity report is an activity score and the sleep report is a sleep score, and the mobile device provides a life score to the user that is based on information used to create the sleep score and activity score.

25. The system of claim 24, wherein the mobile device sends the activity score, sleep score, and life score to a website, and the user can view the activity score, sleep score, and life score on the website.

26. The system of claim 23, wherein the controller includes a microphone that detects the user's snoring, and the controller sends the information related to the user's snoring to the mobile device.

27. The system of claim 23, wherein the tracker has a screen that displays an activity score based on the user's awake movements.

28. The system of claim 23, wherein the controller further includes a speaker that transmits sound via bone conduction.

29. The system of claim 23, wherein the controller further includes a motor that causes the pillow to vibrate.

30. The system of claim 23, wherein the pillow further includes:
an inner top layer and an inner lower layer;
a flexible bladder located between the inner top layer and inner lower layer;
a closure mechanism located in a side of the pillow that can be used to open and close a hole in the side of the pillow;
a plurality of soft members, wherein each member includes a round core and a plurality of projections extending from the core, and the members are configured to be inserted into and removed from the bladder through the hole in the side of the pillow to increase and decrease, respectively, the size of the pillow.

31. A method for scoring a person's activity and sleep habits, comprising:
providing a web-enabled mobile device;
providing a wearable activity tracker that includes a first transceiver and a first sensor, wherein the first sensor detects a user's movement and the first transceiver communicates with the mobile device;
providing a pillow including a controller having a second transceiver and a second sensor, wherein the second transceiver communicates with the mobile device and the second sensor detects a user's movements;
downloading an application from a website with the mobile device in order to facilitate communication between the mobile device and the tracker and the mobile device and the pillow controller;
detecting a user's movements over time while the user is awake with the first sensor of the tracker;
sending the information related to the user's awake movements from the tracker to the mobile device via the first transceiver;
using the application on the mobile device to calculate an activity score for the user based on the awake movement information;
detecting a user's movements over time while the user is asleep with the first sensor of the tracker;
sending the information related to the user's sleep movements from the tracker to the mobile device via the tracker first transceiver;
detecting a user's movements over time while the user is asleep with the second sensor in the pillow controller;
sending the information related to the user's sleep movements from the pillow controller to the mobile device via the second transceiver;
using the application on the mobile device to calculate a sleep score for the user based on the sleep movement information sent by the tracker and by the pillow controller;
using the application on the mobile device to calculate a total life score based on the awake movement information sent by the tracker and on the sleep movement information sent by both the tracker and the pillow controller; and
displaying the activity score, the sleep score, and the total life score on a screen of the mobile device.
